(19) United States
(12) Patent Application Publication
(10) Pub. No.: US 2007/0130674 A1
(43) Pub. Date: Jun. 14, 2007

(54) Eye protection device, particularly against laser beams and high-intensity light beams

(76) Inventor: Antonius Beyer, Waltrop (DE)

Correspondence Address: Edward G. Greive;Renner, Kenner, Greive, Bobak, Taylor & Weber, Fourth Floor, First National Tower, Akron, OH, 44308-1456, US

(21) Appl. No.: 11/378,117

(22) Filed: Mar. 16, 2006

(30) Foreign Application Priority Data
Dec. 9, 2005 (DE) 102005058888.3-55

(51) Int. Cl. A61F 9/02

(52) U.S. Cl. 2/431

(57) ABSTRACT

An eye protection device (1) includes two internally concave and externally convex protective shells (2, 3), which cover the eyes and the eyelids of a wearing person and are connected by an elastic wire bow (8), whose ends (6, 7) are attached to a projection (4, 5) on the protective shells (2, 3) at a distance from their outer edge (2c, 3c). The eye protection device no longer has the sharp-edged mechanical pressure of the outer edge of the protective shells, which feels unpleasant, and nonetheless ensures a secure seal of the outer edge of the protective shells in relation to the facial skin. Each protective shell (2, 3) is completely enclosed on its outer edge (2c, 3c) by an opaque, entropy-elastic ring (9, 10) having high heat resistance and the band-shaped holding unit (14) is provided with an adjustment and locking device (15), using which the untensioned length of the holding unit (14) may be lengthened or shortened rapidly, the holding unit (14) forming a joint spring (9, 10, 14) in connection with the entropy-elastic rings (9, 10) of the protective shells (2, 3), whose spring pre-tension is adapted to the mechanical pressure compatibility of the wearing person via the adjustment and locking device (15).

### TECHNICAL FIELD

The present invention relates to an eye protection device, particularly against laser and light beams, comprising two internally concave and externally convex protective shells, which cover the eyes and the eyelids of a wearing person and are connected by an elastic wire bow, whose ends are attached to a projection on the protective shells at a distance from the outer edge and whose middle part maintains a clear interval to the nose, the protective shells being held on the head of the wearing person using an elastic holding unit.

### BACKGROUND ART

A known eye protection device of this type is known from DE 101 06 668 C2. In this embodiment, the wire bow is made of a thin, break-proof wire, also having high torsion elasticity, made of a titanium-nickel alloy, whose free ends are permanently bonded to the convex outside of the completely closed protective shells by laser welding, soldering, or gluing.

Eye protection devices of this type are used not only in noninvasive interventions on the skin, but rather also in skin treatments through multicolored light of high intensity.

A further eye protection device of the type cited at the beginning is disclosed in U.S. Pat. No. 2,283,752. In this case, the ends of the wire bow are shaped into an eye, which is held on a rivet connection penetrating the particular protective shell in the central area. At the time of publication of this specification, there were not yet any laser beams and therefore also no laser beam operations, but this eye protection device could also have been used for this purpose because its protective shells may be made from metal. This previously known eye protection device is subject to the disadvantage that each protective shell is penetrated by a rivet connection and therefore laterally incident laser beams must be feared. This is particularly true if the eye connections of the ends of the wire bow loosen or expand with continuing use.

A further eye protection device, which is foreign to the species, however, is known from U.S. Pat. No. 5,918,600, which has the same content as WO 89/53775. In this case, the U-shaped, elastic wire bow is connected at its ends to the outer edge of the protective shells via a cylindrical spiral, the spirals being placed on rigid hooks and being permanently connected therewith to an outer edge of the protective shell. Either hooks or attachment projections, which are also wound by a cylindrical spiral and provided with a hook at their ends, are positioned on the diametrically opposite outer edge of the protective shells, on which an elastic band is hooked as a holding unit. This eye protection device is provided with closed protective shells, but is connected to the disadvantage that the wire bow connected rigidly to the hooks via the spirals breaks off relatively easily and for persons having a large eye spacing, the eye area of the protective shells facing away from the wire bow folds outward when the wire bow is bent and must be pulled against the facial area of the affected wearing person surrounding the eyes and the eyelids accordingly by the tensile band with corresponding tensile forces. Punctual strains thus arise due to the attachment hooks on the outer area and also due to the spring hooks on the inner area, which are felt as extremely unpleasant by the affected wearing person during the surgical intervention.

All three embodiments have the shared disadvantage that the hard protective shells made of metal are implemented having sharp edges and “dig into” the facial skin in the area around the eyes with corresponding mechanical pressure. This is found to be extremely unpleasant by the wearing person and may result in this person shifting the protective shells during the operation and a light leak for the entry of damaging laser beams thus arising between the protective shells and the facial skin. Such a light gap may also result because a gap remains open when the outer edges of the hard protective shells are placed on facial wrinkles, scars, warts, or other skin imperfections, resulting in a light leak.

### DISCLOSURE OF THE INVENTION

The present invention is based on the object of providing an eye protection device of the type cited at the beginning, which no longer has the sharp-edged mechanical pressure of the outer edge of the protective shells found to be unpleasant and, nonetheless, ensures a secure seal of the outer edge of the protective shells against leaks and a pleasant-feeling seat of the protective shells on the parts of the facial skin to be sealed.

This object is achieved according to the present invention in connection with the species cited at the beginning in that each protective shell is completely enclosed on its outer edge by an opaque, entropy-elastic ring having high heat resistance and the band-shaped holding unit is provided with an adjustment and locking device, using which the untensioned length of the holding unit may be lengthened or shortened rapidly, the holding unit forming a joint spring in connection with the entropy-elastic rings of the protective shells, whose spring pre-tension is adapted to the pressure compatibility of the wearing person via the adjustment and locking device.

Through this implementation, for the first time, protective shells are provided whose contact position is found pleasant by the wearing person of the entropy-elastic ring and, because of their larger contact surface in relation to the narrow outer edge of the protective shells, the pressure per area unit and therefore contact pressure are reduced and, together with the band-shaped holding unit and the adjustment and locking device, permit a spring pre-tension which, nonetheless, ensures absolute light-tightness without any leaks of the protective shells on the facial skin of the wearing person and may be adapted rapidly to the pressure compatibility of the wearing person.

According to an especially advantageous refinement of the present invention, the entropy-elastic rings have a peripheral groove on their inner edge facing toward the outer edge of the protective shells, which overlaps the outer edge of the protective shells in their final position under entropy-elastic spring pre-tension. A more secure seat of the entropy-elastic rings on the shells, which are made of metal, and an elastic adaptation of the protective shells to skin imperfections is thus ensured.

According to an especially advantageous refinement of the present invention, the entropy-elastic rings for the protective shells are made of a silicone which has a temperature resistance of 200° C. over four hours.

These entropy-elastic rings are sterilizable both using chemical disinfection agents and also using thermal disinfection in an autoclave and may be replaced without tools in the event of wear. The sterility which is absolutely required in the area of operating rooms is thus ensured.

The adjustment and locking device advantageously comprises a spherical or cylindrical body having a central through opening, through which the band-shaped holding unit is pulled twice through a constriction as the adjustment and locking device.

Alternatively, the adjustment and locking device is formed by a baffle projection inside the through opening.

A further spherical or cylindrical body is pulled onto the band-shaped holding unit in order to prevent the band-shaped holding unit from slipping through the adjustment and locking device.

The band of the holding unit advantageously comprises a nonflammable silk, which may only be carbonized, and rubber bands, whose ends are wound into a loop and are held together in a nonadjustable way by a heat-shrinkable sleeve made of a polyolefin having an internal adhesive. A reduction of the spring pre-tension of entropy-elastic rings and holding unit due to undesired expansion of the loop length is thus reliably suppressed.

The nonadjustable loops are each held in a way known per se by a hook which is connected permanently to the protective shells at a distance from the outer edge thereof.

According to an advantageous refinement of the present invention, the ends of the wire bow are mounted by a cylindrical sleeve as a projection of the protective shells in a pivot bearing. This pivot bearing comprises a plastic sleeve made of PTFE (polytetrafluorethylene), which is inserted fixed into a cylindrical hole of the cylindrical sleeve. Since PTFE is a plastic having high thermal resistance having a temperature resistance up to 250° C., the eye protection shells may be thermally sterilized completely in an autoclave with the wire bow after removal of the adjustment and locking device.

In a way known per se, the wire bow comprises a thin, break-proof wire, which also has high torsion elasticity, made of a titanium-nickel alloy. It is implemented as either U-shaped, trapezoidal, triangular, or polygonal in its middle part.

### BRIEF DESCRIPTION OF THE DRAWINGS

An exemplary embodiment of the present invention is illustrated in the drawings.

FIG. 1 shows a top view of the eye protection device,

FIG. 2 shows an enlarged view of the projection on the right eye shell having the end of the wire bow mounted pivotably therein,

FIG. 3 shows an enlarged view of the projection on the left eye shell having the other end of the wire bow mounted therein,

FIG. 4 shows the view of the eye protection device in the direction of arrow IV of FIG. 1,

FIG. 5 shows the front view of an entropy-elastic ring,

FIG. 6 shows the rear view of an entropy-elastic ring having the groove,

FIG. 7 shows a sectional illustration along the line VII/VII of FIG. 6 in an enlarged illustration,

FIG. 8 shows the eye protection device in position on the eye area of a wearing person having the wire bow pivoted toward the nose,

FIG. 9 shows a perspective top view of the eye protection device in the direction of the arrow IX of FIG. 1,

FIG. 10 shows the eye protection device in position on the face area of a wearing person having the wire bow folded away from the nose area in the direction of the forehead,

FIG. 11 shows a diametral section through the adjustment and locking device in the form of a sphere having a constriction area in an enlarged illustration of the area of FIG. 1, and

FIG. 12 shows a further embodiment of the spherical adjustment and locking device in diametral section having a baffle projection for constricting the passage cross-section for the holding unit in an enlarged illustration of the area XII of FIG. 1.

### PREFERRED EMBODIMENT FOR CARRYING OUT THE INVENTION

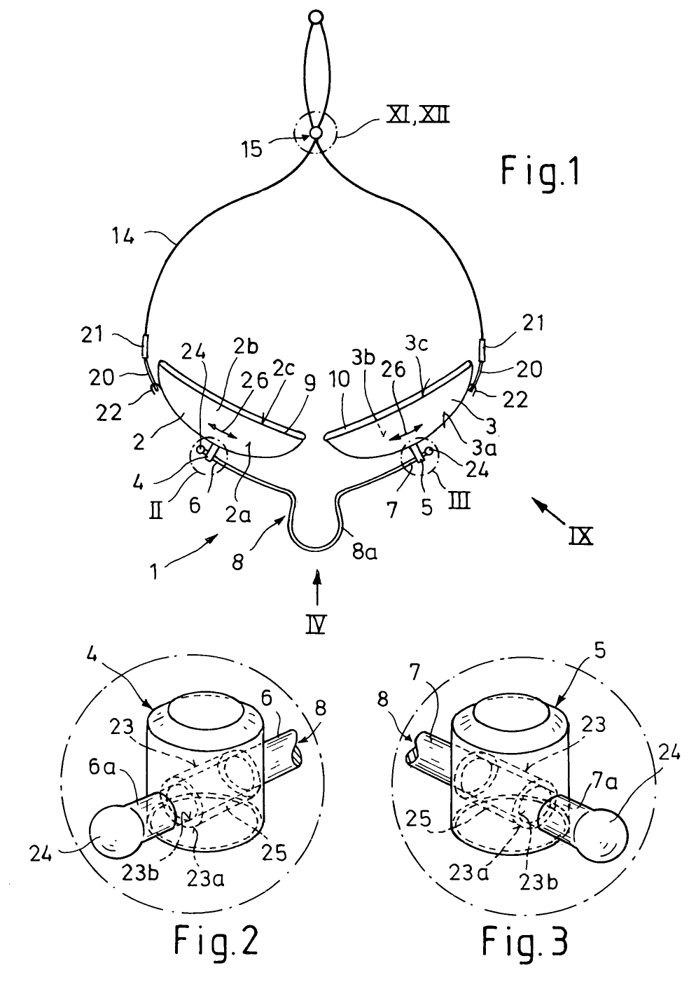

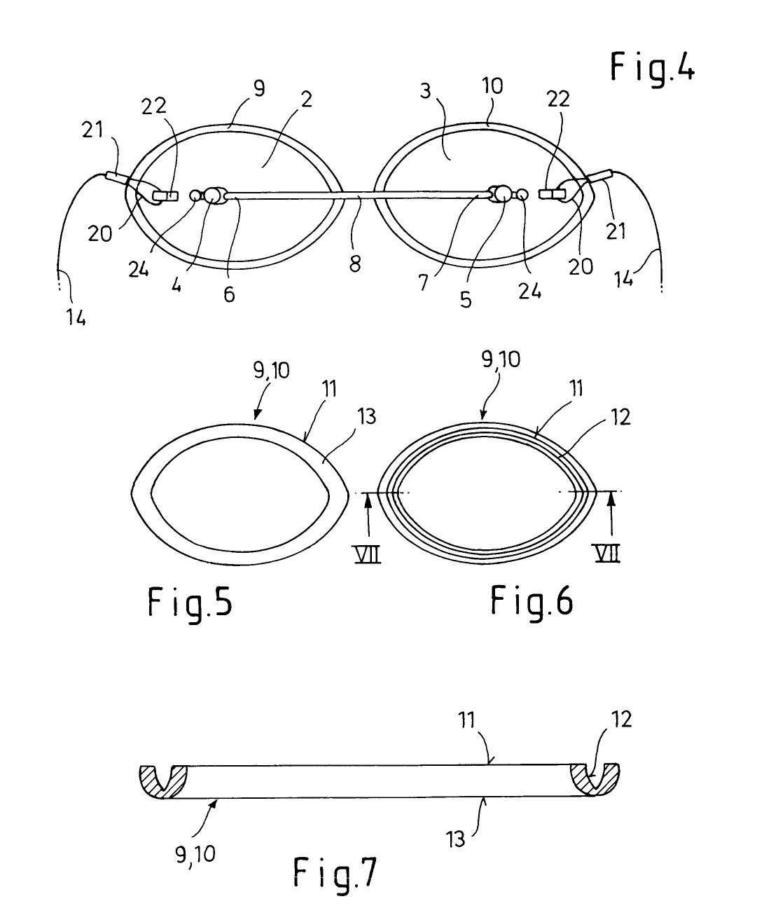

The eye protection device 1 shown in FIGS. 1 and 9 essentially comprises two protective shells 2 and 3 made of metal or ceramic, each of which has a projection 4, 5 in its central area, on which the ends 6, 7 of an entropy-elastic wire bow 8 are pivotably mounted. The protective shells 2, 3 are designed as convex on their outside 2a, 3a and concave on their inside 2b, 3b. The projections 4, 5 have a clear distance from the outer edges 2c and 3c of the protective shells 2 and 3.

Furthermore, the protective shells 2 and 3 are each enclosed on their outer edges 2c and 3c by an entropy-elastic ring 9,10, whose inner edge 11, facing toward the outer edge 2c, 3c is provided with a peripheral groove 12, which overlaps the outer edge 2c, 3c of the protective shells 2, 3 in its final position under entropy-elastic spring tension. The outer edge 13 of the entropy-elastic rings 9,10 is implemented as convex.

The entropy-elastic rings 9,10 for the protective shells 2, 3 comprises a silicone which advantageously has a temperature resistance of 200° C. over four hours. The entropy-elastic rings 9,10 may not only be sterilized in an autoclave, but rather also have been shown to be resistant when they are heated by a laser beam during a surgical intervention.

In addition, the eye protection device 1 is provided with a band-shaped holding unit 14 and an adjustment and locking device 15.

As shown in FIGS. 11 and 12, the adjustment and locking device 15 comprises a spherical or cylindrical body 16 having a central through opening 17, through which the band-shaped holding unit 14 is drawn twice through a constriction which may have a baffle projection 19 within the through opening 17 as shown in FIG. 12. This adjustment and locking device 15 is advantageously made of POM (polyacetal) and has a thermal usage range in air up to 80° C.

The band of the holding unit 14 is advantageously produced from a combination of nonflammable silk or artificial silk, which may only be carbonized, in connection with rubber bands, whose ends are wound into a loop 20 and are held together in a nonadjustable way by a heat-shrinkable sleeve 21 made of a polyolefin having an internal adhesive. The temperature resistance of the heat-checkable sleeve 21 extends from −55° C. to +110° C. and its shrinkage temperature is approximately +120° C.

The loops 20 of the holding unit 14 are pulled onto hooks 22, which are attached to the outside 2a, 3a of the protective shells 2, 3 at a distance A (see FIG. 9) from the outer edge 2c, 3c of the protective shells 2, 3 through welding, soldering, or gluing and are made of the same material as the protective shells 2, 3. The holding unit 14 may thus be removed and, separately therefrom, the protective shells 2, 3 having the wire bow 8 and including the entropy-elastic rings 9, 10 may be sterilized in an autoclave, while in contrast the adjustment and locking device 15 is subjected to a chemical disinfection with the holding unit 14.

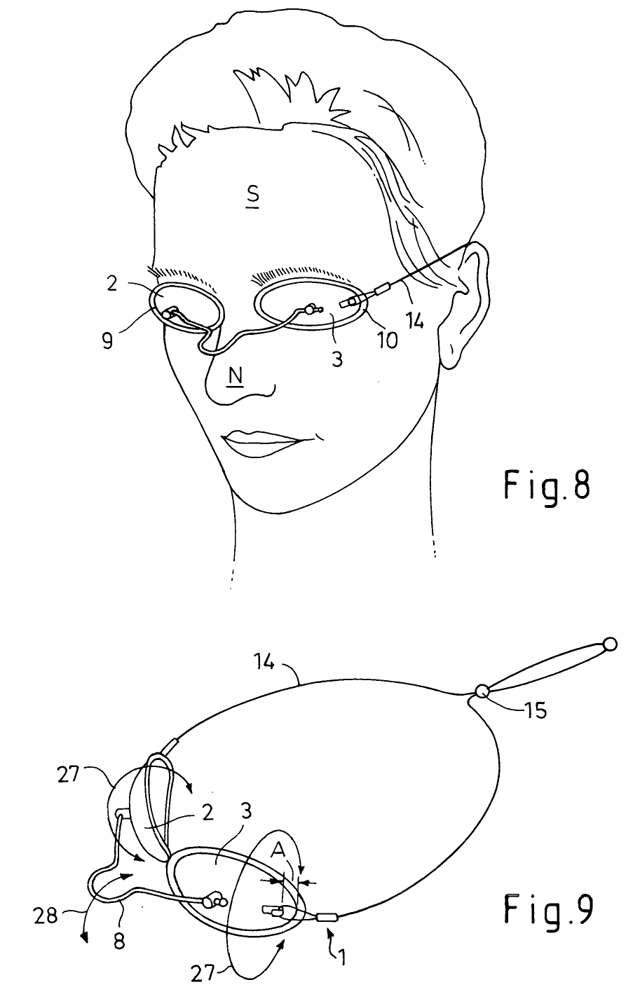

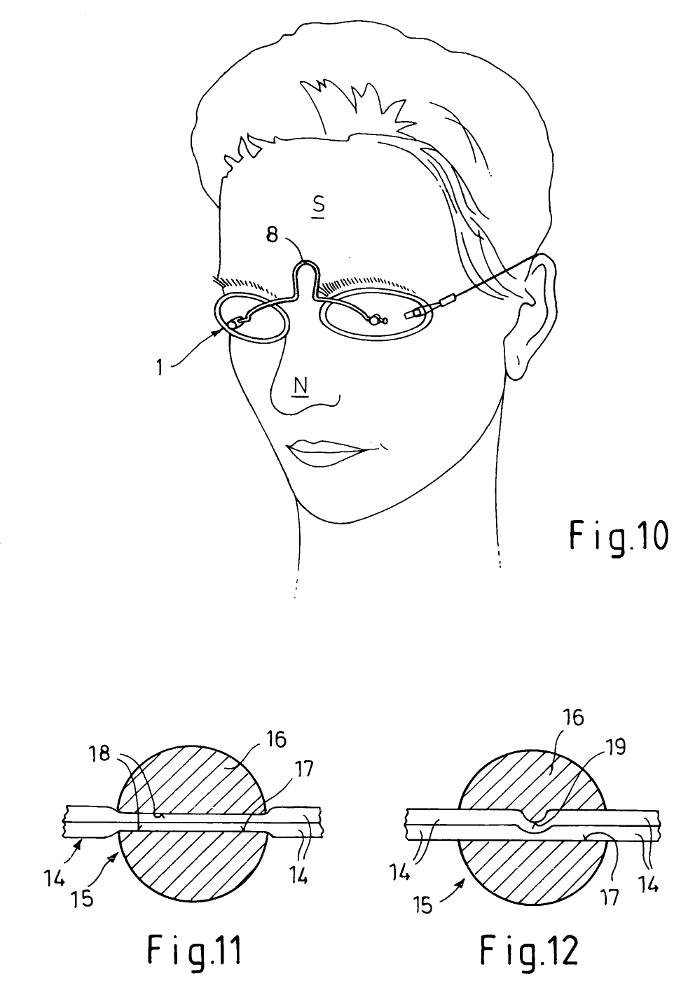

Depending on the area of use of the surgical intervention in the facial area using a laser beam, the wire bow 8 may be an obstruction. For example, if this surgical intervention is to occur in the nose area, the wire bow 8 is folded up to the forehead S of the wearing person as shown in FIG. 10, without contacting the forehead S of the wearing person, however. In contrast, if the surgical intervention using a laser beam occurs in the area of the bridge of the nose or the forehead S of the wearing person, the wire bow 8 is folded down to the nose N as shown in FIG. 8, without contacting the nose N, however. The protective shells 2, 3 held by the holding unit 14 do not have their position impaired upon this pivot movement of the wire bow 8, because the ends 6, 7 of the wire bow 8 are mounted in the pivot bearing 25 of the projections 4, 5. This pivot mounting is also obvious from FIGS. 2 and 3.

As shown in FIG. 2, the end 6 of the wire bow 8 penetrates a stepped cylindrical hole 23, whose part of the hole 23a having a larger diameter faces toward the wire bow 8 and whose part 23b having a smaller diameter faces toward the free end 6a, which is provided with a swelling 24 in order to prevent the end 6 from pulling out of the projection 4. A cylindrical plastic sleeve 25 made of PTFE (polytetrafluorethylene) is inserted fixed in place in the part 23a as a pivot bearing 4, 5, 25 having the larger diameter. The projections 4, 5 in the form of cylindrical sleeves are made of a chrome-nickel alloy which approximately corresponds to the material of the protective shells 2, 3.

The wire bow 8 is produced in a way known per se from a thin, break-proof wire, also having high torsion elasticity, made of a titanium-nickel alloy, in order to prevent the protective shells 2, 3 from falling apart even in the event of extreme torsion strains.

In order to set correct eye spacing of the protective shells 2, 3, the ends 6, 7 of the wire bow 8 may be shifted in both directions of the double arrow 26 shown in FIG. 1 and both the wire bow 8 and also the protective shells 2, 3 may be pivoted in relation to one another in the direction of the double arrows 27, 28 as shown in FIG. 9.

The core of the present invention is that the entropy-elastic rings 9, 10 of the protective shells 2, 3, in connection with the elastic holding unit 14, form a joint spring whose spring pre-tension may be adapted individually to the mechanical pressure compatibility of the wearing person via the adjustment and locking device 15. This is because both metallic and also ceramic materials of the protective shells 2, 3 are subject to the disadvantage that they feel cold when placed on the eye area and therefore must first be heated by the facial area. Because of this unpleasant pressure and temperature feeling, every wearing person compensates for this through mechanical shifting. However, the protective shells 2, 3 may thus in turn be shifted into a position in which a leakage gap may arise between the outer edges 2c, 3c of the protective shells 2, 3 and the facial skin in contact therewith, which lets through a laser beam. This is now prevented by the entropy-elastic rings 9, 10 in connection with the holding unit 14, which is also elastic, because the rings 9, 10, which are advantageously made of silicone, adapt themselves in a pleasant way to the particular facial contour of the wearing person and thus compensate for imperfections such as wrinkles, scars, warts, or the like in the contact area.

Since silicone additionally has outstanding skin compatibility, the contact of the entropy-elastic rings 9, 10 on the skin is found to be pleasant, so that the wearing person may rest in a stable operation position during the surgical intervention.

Finally, the optimum adjustability of the protective shells 2, 3 in the wire bow 8 as shown by the arrows 27, 28 of FIG. 9 and the adjustability of the eye shells 2, 3 on the ends 6, 7 of the wire bow 8 in the direction of the double arrow 26 as shown in FIG. 1 contributes to achieving a correct eye spacing for an optimum seat and therefore to increased safety against light leaks.

### LIST OF REFERENCE NUMBERS

Eye protection device 1
Protective shells 2, 3
Convex outside of the protective shells 2, 3 2a, 3a
Convex inside of the protective shells 2, 3 2b, 3b
Outer edge of the protective shells 2, 3 2c, 3c
Projections 4, 5
Ends of wire bow 8 6, 7
Wire bow 8
Entropy-elastic rings 9, 10
Interior of the entropy-elastic rings 11
Peripheral groove of the entropy-elastic rings 12
Outside of the entropy-elastic rings 13
Holding unit 14
Adjustment and locking device 15
Spherical or cylindrical body of the adjustment and locking device 15 16
Through opening in the adjustment and locking device 15 17
Constriction of the adjustment and locking device 15 18
Baffle projection in the through opening 17 19
Loop of the holding unit 14 20
Heat-shrinkable sleeves on the loop ends 21
Hooks for the holding unit 14 22
Cylindrical hole in sleeves 4, 5 23
Hole having larger diameter 23a
Hole having smaller diameter 23b
Swelling at the ends of the wire bow 8 24
Cylindrical plastic sleeve 25

Double arrows 26, 27, 28
Spacing of the hooks 22 from the outer edge 2c, 3c A
Forehead of the wearing person S
Nose of the wearing person N

What is claimed is:

1. An eye protection device, particularly against laser and light beams, comprising two internally concave and externally convex protective shells, which cover the eyes and the eyelids of the wearing person and are connected by an elastic wire bow, whose ends are attached to a projection at a distance from the outer edge on the protective shells and whose middle part maintains a clear spacing to the nose, the protective shells being held on the head of the wearing person using an elastic holding unit, characterized in that each protective shell (2, 3) is completely enclosed on its outer edge (2c, 3c) by an opaque, entropy-elastic ring (9, 10) having high heat resistance and the band-shaped holding unit (14) is provided with an adjustment and locking device (15), using which the untensioned length of the holding unit (14) may be lengthened or shortened rapidly, the holding unit (14) forming a joint spring (9, 10, 14) in connection with the entropy-elastic rings (9, 10) of the protective shells (2, 3), whose spring pre-tension is adapted to the mechanical pressure compatibility of the wearing person via the adjustment and locking device (15).

2. The eye protection device according to claim 1, characterized in that the entropy-elastic rings (9, 10) have a peripheral groove (12) on their inner edge (11), facing toward the outer edge (2c, 3c) of the protective shells (2, 3), which overlaps the outer edge (2c, 3c) of the protective shells (2, 3) in its final position under an entropy-elastic spring pre-tension.

3. The eye protection device according to claim 1, characterized in that the entropy-elastic rings (9, 10) for the protective shells (2, 3) are made of silicone.

4. The eye protection device according to claim 3, characterized in that the silicone has a temperature resistance of 200° C. over four hours.

5. The eye protection device according to claim 4, characterized in that the entropy-elastic rings (9, 10) are sterilizable in an autoclave.

6. The eye protection device according to claim 1, characterized in that the adjustment and locking device (15) comprises a spherical or cylindrical body (16) having a central through opening (17), through which the band-shaped holding unit (14) is pulled twice through a constriction (18) as the adjustment and locking device (15).

7. The eye protection device according to claim 1, characterized in that the adjustment and locking device (15) is provided with a baffle projection (19) inside the through opening (17).

8. The eye protection device according to claim 1, characterized in that the adjustment and locking device (15) is made of POM (polyacetal) and has a thermal usage range in air up to 80° C.

9. The eye protection device according to claim 1, characterized in that the band of the holding unit (14) is made of a combination of nonflammable silk, which may only be carbonized, and rubber bands, whose ends are wound into a loop (20) and are held together nonadjustably using a heat-shrinkable sleeve (21) made of a polyolefin having an internal adhesive.

10. The eye protection device according to claim 9, characterized in that the temperature resistance of the heat-shrinkable sleeve (21) extends from −55° C. to +110° C. and its shrinkage temperature is +120° C.

11. The eye protection device according to claim 9, characterized in that the nonadjustable loops (20) are held in a way known per se by a hook (22), which is connected permanently to the protective shells (2, 3) at a distance from the external edge (2c, 3c) thereof.

12. The eye protection device according to claim 1, characterized in that the ends (6, 7) of the wire bow (8) are mounted in a cylindrical sleeve (4, 5) as a projection of the protective shells (2, 3) in a pivot bearing (4, 5, 25).

13. The eye protection device according to claim 12, characterized in that the pivot bearing (4, 5, 25) comprises a plastic sleeve (25) made of PTFE (polytetrafluorethylene), which is inserted fixed into a cylindrical hole (23a) of the cylindrical sleeve (4, 5).

14. The eye protection device according to claim 13, characterized in that the cylindrical hole (23) is stepped and the plastic sleeve (25) is inserted into the part of the hole (23a) having a larger diameter and the end (6, 7) of the wire bow (8) is guided in the part (23b) having a smaller diameter and is provided on its free end (6a, 7a) with a swelling (24) which exceeds the smaller diameter of the hole (23b).

15. The eye protection device according to claim 12, characterized in that the cylindrical sleeves (4, 5) are made of a chrome-nickel alloy and are soldered, welded, or glued to approximately the middle of the protective shells (2, 3).

16. The eye protection device according to claim 1, characterized in that the wire bow (8) is made in way known per se of a thin, break-proof wire, which also has high torsion elasticity, made of a titanium-nickel alloy.

17. The eye protection device according to claim 1, characterized in that the wire bow (8) is implemented as U-shaped, trapezoidal, triangular, or polygonal in its middle part.
